(19) United States
(12) Patent Application Publication
(10) Pub. No.: US 2002/0147424 A1
(43) Pub. Date: Oct. 10, 2002

(54) Transdermal magnetic drug delivery system and method

(76) Inventors: Alvin Ostrow, Ra'anana (IL); Joseph Tannenbaum, Jerusalem (IL); Mark Fenster, Ra'anana (IL)

Correspondence Address: Edward Langer, Pat. Atty., c/o Landon & Stark Associates, One Crystal Park, Suite 210, 2011 Crystal Drive, Arlington, VA, 22202, US

(21) Appl. No.: 10/025,971

(22) Filed: Dec. 26, 2001

Related U.S. Application Data
(60) Provisional application No. 60/317,163, filed on Sep. 6, 2001.
(60) Provisional application No. 60/257,290, filed on Dec. 26, 2000.

(51) Int. Cl. A61N 1/30

(52) U.S. Cl. 604/20; 600/9

(57) ABSTRACT

A transdermal magnetic drug delivery system and method which can deliver, via a novel electrode design, multiple drugs in a controlled manner using a variety of methods including magnetophoreris, iontophoresis, sonophoresis, photophoresis and others. The combined use of these methods not only enhances transport but also allows for the optimum parameters for drug delivery to be realized. The drugs may be manipulated, tagged or doped with a magnetic carrire to increase their susceptibility to magnetophoresis and to provide a method for tracking their absorption in conjunction with biosensing.

### FIELD OF THE INVENTION

The present invention relates to a transdermal drug delivery system employing magnetic fields in singular or in combination with other methods to transport various substances of varying molecular weights and magnetic susceptibilities across the skin, an artificial membrane or biological barrier via a novel electrode design. According to the method of the present invention, these substances may be manipulated, tagged or doped so as increase their susceptibility to magnetophoresis and to use them for biosensing.

### BACKGROUND OF THE INVENTION

Controlled delivery of drugs through the skin by use of transdermal patches is well known in the prior art. Passive transdermal drug delivery patches provide advantages over other drug delivery method by delivering the drug directly to the affected area. This method is advantageous over other known methods such as oral administration, which necessitates absorption through the digestive tract, or Intravenous (IV) drug administration, which involves needles. Currently, certain patient types present serious problems to traditional IV techniques. These patients include: patients with blood disorders, immuno-compromised patients, patients with renal dysfunction, patients with vein disorders or deep set veins and small children. It is estimated that patients in the above categories represent at least 20-25% of all hospital patients. Both oral and intravenous administration involve administering high doses of drug to the body at one time, systemically affecting the whole body with the pharmaceutical. These high levels of drug concentration in the blood can create toxic side effects. In addition, only a very small percentage of the drug reaches the affected target area in the body.

There has been a trend toward demands for new methods of self-administered prescription pharmaceuticals such as time-release oral medications and transdermal patches. Transdermal delivery provides medication specifically to the area of treatment. However, the number of passive transdermal drug delivery patches available, such as the nicotine, estrogen and nitroglycerine patches, are limited because they are effective only with small-molecule drugs. Many of the newly developed proteins and peptide drugs are too large to be delivered through passive transdermal patches, forcing pharmaceutical companies to seek advanced delivery technology such as electrical assist (termed interchangeably either iontophoresis or electrophoresis) for large-molecule drugs.

Iontophoresis is a technique employed for enhancing the flux of ionized substances through membranes by application of electric current. The principal mechanisms by which iontophoresis enhances molecular transport across the skin are (a) repelling a charged ion from an electrode of the same charge, (b) electroosmosis (the convective movement of solvent that occurs through a charged pore in response to the preferential passage of counter-ions when an electric field is applied) and (c) increasing skin permeability due to application of electrical current.

It is known that with the continued passage of electrical current through the skin, the skin undergoes adaptation to the current and its impedance rises. Typically this develops heat, as the current is continuously provided and cannot penetrate the impedance of the skin. Since there is no penetration of the current, no drug delivery can occur. At an extreme level, ionic shift occurs affecting the pH of the skin, which may lead to burning of the skin,

In order to allow the drug to penetrate the skin and especially the stratum corneum, artificial pores are created by an electric current via a transdermal electrode in a process called electroporation. Prior art systems use a flat electrode applied to the skin to cause electroporation. This method provides pores that appear in an uncontrolled random fashion. One factor in the unreliable formation of the pores is their dependence on proper surface contact of the electrode with the skin. Another problem is differing surface electrical resistance of different areas of the skin. Because of the random appearance, it can happen that two pores are formed adjacent to one another, such that the opening is too large to function as a pore and becomes a useless leak. Another consequence of the randomness of the flat electrode is that some parts of the skin may receive a very high and therefore harmful current density while other parts receive a very low and ineffective current density.

The artificial pores caused by electroporation are not stable and shrink after a short time. This markedly downgrades the efficiency of the electroporation technique.

Many drugs are currently under development and also have been formulated for commercial use in the pharmaceutical Industry employing iontophoresis. Both passive and electrically assisted transdermal drug delivery necessitate wearing transdermal patches made of synthetic substances consisting of a high co-polymer content for long periods of time, often causing skin reactions due to the body's rejection of the membrane as being foreign to the skin. Also, most membrane patches require a specific drug designed toward use with a particular membrane for a specified limited time of usage.

A major drawback in iontophoresis is the limitation on the size of the substance which can be driven across the skin. Many drugs currently in use have very high molecular weight (e,g. insulin—M.W. 6000) and this poses a challenge to iontophoresis.

Other sources besides electric fields have been used to cause ionization so as to move molecules including magnetic fields, ultrasound and even light. These can be used alone or in combination to more efficiently move drugs across a membrane

The following background material is designed to provide the reader with an introduction to magnetic theory so as to understand the present invention more clearly. The following topics are discussed below:

I. Diffusion in a Magnetic Field

II. Magnetic Forces in Heterogeneous Media

III. Drug Transport

IV. Diamagnetic Transport Hypothesis

V. Types of Magnets and Magnetic Fields

VI. Magnetophoresis

VII. The Membrane and Magnetic Drug Delivery

VIII. Electromagnetophoresis

IX. Electrical Field Versus Magnetic Field

X. Diamagnetic or Paramagnetic Definition of a Substance

1. Diffusion in a Magnetic Field

The mathematical equation for concentration of molecules driven by diffusion and the magnetic force is represented by the following formula:

c/t=D²/x²/x*[F(x)/c] (1)

F(x)=magnetic force

c=concentration

t=time

x=distance

D=diffusion constant

The diffusion equation in a magnetic field shows the concentration versus time and distance. As shown in FIG. 1, concentration change due to the magnetic force on gamma-globulin molecules in strongly paramagnetic media was detected experimentally by the method of optic interferometry. The depiction in FIG. 1 is a drawing of the lines of optical interferation.

The magnetic effect using a flow loop with a section of alternating magnetic exposure is maximized at particular velocities. The flow loop has several pairs of permanent magnets with north and south poles facing each other, and placed alternately along a tube. In these loops, the fluid flows through the section instantaneously, so that the pulse-like magnetic field is applied to the flowing fluid,

II. Magnetic Forces in Heterogeneous Media

A gradient magnetic field causes the gradient of pressure between two parts of a solution contained in different concentrations of soluble substances, thus modulating the diffusion molecules through the membrane. For active membranes, e.g., cell membranes, capable of amplification of physical stimuli with the effect of magnetic force, it becomes difficult to predict the result. Therefore it must be evaluated and substantiated by controlled scientific experimentation. With the inducement of a magnetic force employing higher gradient magnetic field (HGMF), a mass transfer of molecules across the membrane is produced, and mass transfer of molecules is increased in proportion to the intensity of the gradient magnetic field employed.

III. Drug Transport

All elements, substances and drugs have been found to have magnetic susceptibilities. These susceptibilities can be classified into three categories: ferromagnetic, paramagnetic, and diamagnetic, depending on their affinity towards a magnetic field. The ferromagnetic substances show maximum acceptability of the magnetic field lines. The paramagnetic substances take up a position parallel to the magnetic field and act as a micromagnets. In the case of diamagnetic substances, the affinity exists in a lesser amount.

Two basic mechanisms of the effects of a magnetic field are involved in the mechanism of drug transport in a model system:

(1) diffusion of molecules due to the magnetic field, and

(2) the mechanical stress at the border of materials of different magnetic susceptibilities,

Both mechanisms are responsible for the transport of drug molecules under the influence of a magnetic field. Magnetic susceptibility of biological tissues and drugs provides the scientific basis for theoretically predicting the magnetokinetic effect of magnetic fields on magnetic properties and characteristics of molecules. Therefore, magnetic properties of (drug) molecules and their physical characteristics should be comparatively evaluated within the model system being experimented on while under exposure of an induced magnetic field,

Magnetophoresis, as defined in this application, is the use of any static or electromagnetic singular or combined AC and DC magnetic fields influencing movement of substances in solute form to induce magnetoporation by magnetokinesis, to actively diffuse or permeate a substance or substances across a biological barrier, i.e. membranes and skin.

Murthy SN M.S. Ramaiah College of Pharmacy, Bangolore India published an article “Magnetophoresis: an approach to enhance transdermal drug diffusion” in Pharmazie 54 (1999) 5, incorporated herein by reference. It was reported that benzoic acid, a diamagnetic substance, had enhanced drug diffusion across rat abdominal skin due to the influence of a magnetic field. The experiment was performed with alternating on-off fields in the same diffusion set-up, and results confirmed that the difference in flux between passive and magnetic diffusion is not due to any variation in the experimental condition or membrane properties, The influence of magnetic field strength on diffusion flux was determined and was found to increase with increasing applied field strength.

IV. Diamagnetic Transport Hypothesis

Diamagnetism is the phenomenon of a magnetic field inducing a magnetic field that opposes it in a material. In other words, a diamagnetic material has a negative susceptibility. (“Diamagnetic Susceptibilities” by Thayer Watkins San Jose State University, Internet Search, June 2000). A diamagnetic substance either is repelled from the field or it moves from a field of higher strength to a region of lower strength.

When an electron moving in an atomic orbit is in a magnetic field B, the force exerted on the electron produces a small change in the orbital motion, causing the electron to orbit about the direction of B. As a result, each electron acquires additional angular momentum that contributes to the magnetization of the sample. The susceptibility is given by

X=−μ₀N (e²/6m)Σ<r>² (2)

where the symbol Σ<r>² means “the sum of’ <r>², where <r> is the sum of the mean square radii of all electron orbits in each atom, e and m are the charge and mass of the electron, and N is the number of atoms per unit volume. The negative sign of this susceptibility is a direct consequence of Lenz's law. When B is switched on, the change in motion of each orbit is equivalent to an induced circulating electric current in such a direction that its own magnetic flux opposes the change in magnetic flux through the orbit; i.e., the induced magnetic moment is directed opposite to B.

Diamagnetism is a property that arises from the interaction of paired electrons with the magnetic field and therefore is an inherent property of matter irrespective of whether it also contains unpaired electrons. In the case of substances containing unpaired electrons, the paramagnetism predominates and hence overshadow the diamagnetism. Diamagnetism is an induced effect and exists as long as the magnetic field is applied, without leading to permanent molecular changes of the substances being magnetokinetically influenced. Murthy's magnetophoresis experiment was based upon the assumption that in comparison with “passive diffusion”, application of a magnetic field would exert a force of repulsion on the diamagnetic substance (benzoic acid) creating “magnetic diffusion” and would thus help enhance diffusion across a biological membrane.

V. Types of Magnets and Magnetic Fields

Any substance has magnetic properties, characterized by the magnetic susceptibility (κ). A substance moves in a non-homogeneous field based on the value of κ.

If κ<0 the compound is diamagnetic (repelled from the area of HGMF).

If κ>0 the compound is paramagnetic (or ferromagnetic, κ>>O) and attracted to the High Gradient Magnetic Fields (HGMF). Drugs must be stronger diamagnetic than the liquid medium, HGMFs are shown in FIG. 2.

Diamagnetic compounds are repelled from a stronger magnetic field by a ponderomotive force Fₘ, according to the following equation;

Fₘ=(Δκ)V Igrad(H²/2)| (3)

where Δκ is the difference of magnetic susceptibilities between the drug and solution,

For example, with a starch particle as a diamagnetic body, the magnetic force is equal to the gravity force, if the dynamic factor grad (H²/2) of the field equals:

grad(H²/2)=10⁹−10¹⁰Oe²/cm or (4)

grad(B₀²/2)=˜1400T²/m (5)

VI. Magnetophoresis

Recently developed techniques using superconducting magnets have revealed several phenomena of effects of strong magnetic fields on materials. It has been reported that fibrin polymers in gradient magnetic fields drift in a specific direction, and concentrations of the fibrin change. When a solution containing water and diamagnetic macromolecules is exposed to gradient fields, magnetic forces act on the water and diamagnetic macromolecules. Diamagnetic molecules in water drift in a specific direction due to the difference in the diamagnetic susceptibility of the molecules and water. The results indicate that “magnetophoresis” of biological molecules occurs in gradient magnetic fields of up to 8 T and 50 T/m.

The use of magnetophoresis for paramagnetic/ferromagnetic technologies was developed in the 1970's (Kutznetsov, A). More recently, the use of magnetophoresis for blood diagnosis has been researched in the U.S. (Zborowsi M. Anal, Chemistry 1995, 67, 3702-3712,). Presently, magnetic drug delivery is performed using ferromagnetic materials from the “magnetic carriers” industry.

VII. The Membrane and Magnetic Drug Delivery

Modulation and optimization of drug release from magnetically controlled polyrneric drug delivery devices has been studied. Release rates from drug polymer matrices embedded with small electromagnets significantly increase in the presence of oscillating magnetic fields. Thus, electromagnets may be used as a means to optimize externally regulated controlled release systems. This concept is desirable since there is a need to change or modulate the release rate on demand, once release as commenced (Hseih OS et al. Proc. Natl. Acad. Sci. Vol. 78, No 3, pp. 1863-1887, March 1981). The formula for the magnetic forces against distance is F˜1/Dⁿ, where D is the distance (magnetic force is inversely proportional to the distance, n is a number typically greater than 3).

Findings by Biokhra R L and Joshi J (1999) in the Journal of Colloid and Interface Sciences 220, 458-464 show that flow through membranes is affected by magnetic fields.

Magnetic filters can be constructed that will extract micron-sized paramagnetic particles from a fluid passing through a filter (Watson J H, J. Applied Phys., vol. 44, No. 9, September 1973).

Thus, optimum parameters for magnetophoretic drug delivery may include:

i. magnetically, ferromagnetically or paramagnetically treated substances in a solute;

ii. membranes made to suit paramagnetic properties; and

iii. effects of all variables described above of magnetic drug effect on transport

Extensive research has led to the assertion that to transfer magnetokinetically diamagnetic solutes through a membrane, high gradient magnetic fields are required. The reason is that the effect of repulsion of the diamagnetic substance is very weak. To increase the magnetic force, a membrane/solution can be embedded/treated with ferromagnatic/paramagnetic elements such as metals, paramagnetic ions, etc. (Coronado E., et al. Advanced Materials for Optics and Electronics, vol.8, Issue 2, 1998, p. 61-76).

The magnetokinetic force required to magnetophoretically permeate substances increases with increasing the gradient of magnetic field. The gradient is greater for small-sized than great-sized ferromagnetic elements as sources of magnetic field (Edelman, E R et al. J. Biomed Mater res. Mar. 21, 1987; (3):339-53, Biomaterials 1993, Vol 14, No. 8).

Thus, manipulation of the solution and the membrane can be researched to provide improved ion transfer by increasing magnetic force which can be yielded by ferromagnetic elements of a small diameter and without highly intensive electromagnets (e.g., superconductive magnets).

Instrumentation is necessary for measuring magnetophoretic mobility of solutions whereby particles of a given composition, size and distribution can be separated. The use of an apparatus for measuring multiparameter signals, including in part, magnetophoretic mobility, is also warranted.

There are many variables that require research and examination to produce magnetophoresis. Among these are:

1. Use of high gradient magnetic fields

2. Use of pulsed AC and DC magnetic fields

3. Size and strength of the permanent magnet

4. Design of membranes conducive to the “Magnetophoretic effect”

5. Design of solutes with paramagnetic, ferromagnetic and diamagnetic substances.

6. Developing proof of concept with various test cell models demonstrating magnetophoresis and/or electromagnetophoresis donor-receiving permanent transport phenomenon.

7. Design of electromagnet for influence on drug delivery by understanding the molecular attenuation point and magnetic susceptibility of substances.

8. Study of electrical impedance of skin: the lower the resistance of the skin over various anatomical areas, the more easily the drugs permeate, The magnetic field seems to have an effect on skin and its electrical properties that affect transdermal drug delivery. For example, it is hypothesized based upon the scientific literature, that acupuncture points have lowered electrical resistance and may prove to be preferred target areas for transdermal transport.

Prior art used oscillating magnetic fields to enhance drug delivery through a biological barrier. This was inefficient and not very portage.

VIII. Electromagnetophoresis (EMP)

The magnetic equation for transdermal drug delivery alone does not seem to be sufficient to create a superior drug delivery device. Rather, it can be used as an addition or enhancement to iontophoretic/electrophoretic transdermnal drug delivery by crossing electric and magnetic and electromagnetic fields to overcome problems with current day state of the art iontophoretic/electrophoratic drug delivery systems.

Electromagnetophoresis was first discovered by Kolin (University of Chicago, USA) in 1952 for other purposes than drug delivery, who originated the theory of “the electromagnetokinetic effect” Leenov and Kolin, Journal of Chemical Physics Vol. 22 No. 4 (April 1954) “Theory of Electromagnetophoresis (i) Magnetohydrodynamic forces experience by spherical and symmetrically oriented cylindrical particles”.

Electromagnetophoresis experiments were conducted at the Technion (Israel) in the 70's by Zvi Karni. According to Karni Z, in “Magnetoelectrophoresis”, Medical and Biological Engineering (May 1975), the effect of a magnetic field has a direct effect on the ionic transport and its flow through viscous media such as saturated polymers, membranes, etc, Karni reports that a magnetic field, when applied perpendicular to the electric field that induces electrophoresis, has a direct effect on the conductivity of the buffer solution and on the ionic fluidity through saturated polymeric electrophoretic strips.

According to Kolin A. in Science, Vol. 117 “An Electromagnetokinetic Phenomenon Involving Migration of Neutral Particles” electrically neutral particles migrate in a magnetic field transversed by an electric current. The migration is perpendicular to the current and to the homogenous magnetic field that is maintained at right angles to the current. If the electrical conductivity of the particles exceeds that of the surrounding conductive fluid, the particles migrate in the direction of the force exerted in the magnetic field upon the current. Particles of lesser conductivity than that of the surrounding field migrate in the opposite direction, whereas particles experience no force if their electric conductivity is equal to that of their environment,

The force of gravity, as well as the form of buoyancy exerted upon a suspended body, can be neutralized. For instance, an air bubble will not rise in acidulated water placed in a horizontal magnetic field of 10,000 oersteds transversed by a perpendicular horizontal current of 1 amp/cm².

R A Mills in Bulletin of Mathematical Biophysics (volume 30, 1968 ), discussed that when uncharged particles are suspended in a conducting fluid, it is usually found that mutually perpendicular electric and magnetic fields applied to the system will give rise to movement of these particles relative to the field. The motion is in a direction normal to each of the applied fields and has been termed “electromagnetophoresis.” Electromagnetophoresis occurs when certain properties of the particles, in particular their electrical conductivity, differ from those of the medium which they are suspended.

IX. Electrical Field Versus Magnetic Field

Problems associated with crossing magnetic and electric fields are created when the fields transverse simultaneously at the same moment, which cancels the fields.

Gunter, Jr. et al. in the text entitled Biophysics of Structure and Mechanism (1978, p.87-95) discusses “trajectories or particles suspended in electrolytes under the influence of crossed electric and magnetic fields” conducted in an electrophoresis chamber. G. Ruhenstroth-Bauer reported “the influence of combined electric and magnetic fields on Biological cells and other particles” Haematologia (1-4) pp 517-521 (1974), Liboff, R L provided a theoretical explanation of this phenomenon entitled “Brownian motion of Chared Particles in Crossed Electric and Magnetic Fields” Physical Review Vol. 141, Number 1 (January 1966).

Thus, it is thought that the same physical laws apply to endogenous drug delivery using compounds with ferromagnetic/paramagnetlc properties, and it may be assumed that the more a substance has a metallic content, either paramagnetic or diamagnetic, the more amenable the substance is to magnetokinetic forces applied by HGMF to actively transport drugs through a membrane and transdermally, as shown in FIG. 3.

X. Diamagnetic or Paramagnetic Definition of a Substance

In order to test different diamagnetic substances by magnetophoresis, the following steps must be taken:

1. Find the quantitative relation between the magnetic power (B/H) and ion transfer via the membrane.

2. Design and develop a generator for the high density electromagnets.

3. Quantitative analysis of the simultaneous activation of magnetophoresis and electrophoresis.

4. Quantitative analysis of the simultaneous activation of magnetophoresis and pulsating electrophoresis.

There are four manipulations to achieve transdermal drug delivery. (These can be for endogenous or exogenous applications:

1. defining power source (electromagnetic vs. magnetic)

2. defining the substance to be transported (as either inherently diamagnetic or paramagnetic—the percentage of metallic compound in the composition of a drug or substance will determine its paramagnetic tendency)

3. defining the membrane—increasing magnetic delivery by treating with magnetic, paramagnetic and diamagnetic properties as required.

4. manipulation of skin to lower resistance and impedance to improve permeation.

Thus, it would be desirable to provide a transdermal magnetic drug delivery system capable of delivering multiple treatment and drug delivery protocols for in vivo use and for controlled targeted drug loop systems by employing the magnetic susceptibilities of pharmaceutical compounds, substances and metals, and that would allow tagging of substances such as drugs for control and delivery modulation.

### SUMMARY OF THE INVENTION

Accordingly, it is a broad object of the present invention to overcome the disadvantages of the prior art and provide a transdermal magnetic drug delivery system and method which can deliver multiple drugs in a controlled manner using a variety of active transport methods including magnetophoresis, iontophoresis, sonophoresis, photophoresis and others. The combined use of these methods not only enhances transport but also allows for the optimum parameters for drug delivery to be realized. The drugs may be tagged with a magnetic carrier to increase their susceptibility to magnetophoresis and to provide a method for tracking their absorption.

In accordance with a preferred embodiment of the present invention there is provided a system for transdermal magnetic delivery of a substance in solution to an acceptor, the system comprising:

at least one substance delivery means employing electroporation of the acceptor in the presence of a magnetic field, and also employing, in sequential fashion, a mode for active transport; and

means for controlling the at least one substance delivery means;

the system being operable to provide combined electroporation of the acceptor and active transport of the substance in solution, in a controlled fashion, thereby delivering the substance to the acceptor,

In another preferred embodiment of the present invention there is provided a method for performing transdermal magnetic drug delivery.

in yet another preferred embodiment of the present invention there is provided a test cell system for determining the optimum parameters for drug delivery.

In still another preferred embodiment of the present invention there is provided a multi-channel system for transdermal magnetic drug delivery using multiple drugs and multiple active transport modes.

The transdermal magnetic drug delivery system (TMDDS) of the present invention is based on the effect of the physical force of magnetic fields, which causes a mechanical stress at the borderline of lesser or greater magnetic substances according to their magnetic susceptibilities. Drugs, according to their magnetic susceptibilities, are either classified as diamagnetic, paramagnetic or ferromagnetic substances. In complex liquids, the mechanical stress generates a convective flow. Mass transfer of fluids is proportionately transported according to the different energy of their components placed at different locations within the heterogeneous magnetic field. Physical forces applied from magnetic fields can be generated for influencing and modulating drug release of substances according to their magnetic susceptibilities.

The combination of iontophoresis, electroporation and magnetophoresis comprise the IEMP theory of ionic drive in which the combination of an electric field and a magnetic field applied to molecules in a solution, activates the affected molecules for transport. By using this method, the brownian motion of the molecules in the solute Is excited by the activation causing a directional convective movement effectively uniformly transporting a concentration of molecules in the activating field. The activating IEMP field induces a higher state of entropy of the targeted molecules while concurrently controlling the direction of molecular transport,

In prior art methods of iontophoresis and electrophoresis, the molecules are dispersed in a random and uncontrolled fashion, whereas in the system of the present invention the molecules are targeted in a straight-line trajectory by means of the combined electric and magnetic fields. This results in a controlled targeted drug delivery system.

Thus, the magnetokinetic concept of magnetic drug delivery release and transport, can be considered for in vivo or in vitro applications, and is a system that includes the variables of magnetic properties of drugs, biological tissues and supporting fluids as component of the magnetic system.

The magnetic field penetrates the skin more effectively, as it is known that the skin has a lower resistance to it, so that an IEMP system requires less current, and therefore saves power and decreases the danger of ionic shift.

In addition, magnetic fields increase permeation through the stratum corneum by the resonance effect, to assist in the magnetoporation of the pores of the skin,

Optimum parameters must be employed for creating magnetohydrodynamic influences generated from magnetic and electromagnetic fields of varying strengths on fluids or solutes that have dissolved elements or substances of various magnetic susceptibilities, atomic weights and sizes, and electrical charges. These influences are used to permeate those dissolved elements or substances through a biological barrier of various types of membranes and thereafter human skin in contact with the membrane and solute being permeated, thus accomplishing “magnetoporation” via synthetic and biological membranes within the drug delivery system of the present invention.

As seen in section I of the Background and in FIG. 1, concentration change can be driven by a pulse-like magnetic field. Thus, in the transdermal magnetic drug delivery system of the present invention, pulse and alternating fields are employed with a concentrated, specifically focused higher gradient magnetic field (HGMF) for more efficacious magnetokinetic transport. The use of fast pulsed and alternating fields eliminates the need for oscillating magnetic fields, and improves the efficiency and portability of the design.

Although magnetic and electric fields normally cancel when the fields cross (see section IX of the Background of the Invention), an extremely fast alternating “duty cycle” solves this problem. This type of cycle means that alternating AC/DC magnetic and or electromagnetic fields when combined with electric fields are timed so as not to transverse without losing optimum effects. This is a refinement of the prior art oscillation methods that were crude and ineffective. The fast duty cycle between electrophoretic moments and magnetic moments produces optimum high energizing forces to excite the electromagnetoporation of ions through a conduit membrane and human epidermal membrane.

To accomplish the above manipulations of the variables that cause drug transport via magnetic means (see section X of the Background), the electromagnet is engineered to modulate the output of matching magnetic field frequencies. These frequencies resonate at varying wavelengths to match the attenuation points relevant to the molecules of the drug in solution, according to their magnetic susceptibilities.

The rise time of the pulse used to generate the magnetic field is coordinated with the speed of ion movement, which is dependent on the molecular structure/molecular weight of the drug substance.

All substances have either weak or strong magnetic susceptibilities according to their composition, however, when they are not intentionally manipulated or doped, i.e. in their natural state, they are termed “neutral”. Experiments have proven that drugs can be transported across a barrier (an artificial membrane or skin) while drugs are “neutral” within a model system.

The inventors have found that in order to improve the controllability, targeting and regulation of drugs within the magnetic drug delivery system so that it can it transport of drugs, either neutral or tagged with magnetic carriers, according to their magnetic susceptibilities, it is necessary to manipulate the substance being delivered. When a substance is intentionally bonded and tagged with a magnetic carrier that is paramagnetic it is termed “manipulated”. Manipulation allows for greater control and target delivery within a model system. This provides a safe method of tagging, avoiding the use of radioactivity, which is currently used as a tag and has serious health disadvantages.

Commercial membranes or novel membranes can be designed and used for transdermal drug delivery, and can be manipulated to become part of a model system for enhancing and increasing magnetic drug delivery transport across a permeable or semi-permeable barrier.

Most pharmaceuticals and/or substances have problems with solubility of varying degrees. Drug transport is facilitated by adding solvents or ilpolytic carriers to unsoluble drug compounds and substances. Using this method, the drug or substance transported can be made to permeate more easily, and this enhances transport across skin. Therefore, the skin can be made to undergo chemical manipulation, including the use of antigens, within a model system.

Applications of the present invention include therapeutic treatment modes involving enhanced drug transport and membrane permeability to diseased or healthy cells, by techniques involving magnetochemotherapy, eletrochemotherapy and magneto-electrochemotherapy. These techniques can be classified under the general field of electro-magnetochemistry.

This system may be used in conjunction with the electrophoretic cuff apparatus drug delivery system of one of the present co-inventors, as described in U.S. Pat. Nos. 5,823,989 and 5,983,134, hereby incorporated in their entireties.

Other features and advantages of the invention will become apparent from the following drawings and the description.

### BRIEF DESCRIPTION OF THE DRAWINGS

For a better understanding of the invention with regard to the embodiments thereof, reference is made to the accompanying drawings, in which like numerals designate corresponding elements or sections throughout and in which:

FIG. 1 shows the lines of optical interferation in a concentration change due to the magnetic force on gamma-globulin molecules in strongly paramagnetic media (prior art);

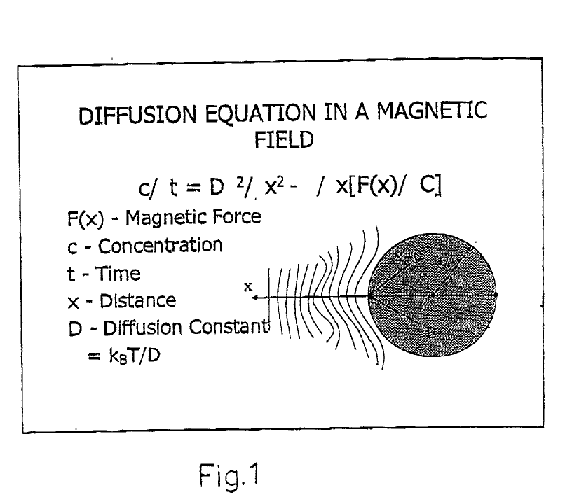

FIG. 2 shows various high gradient magnetic fields;

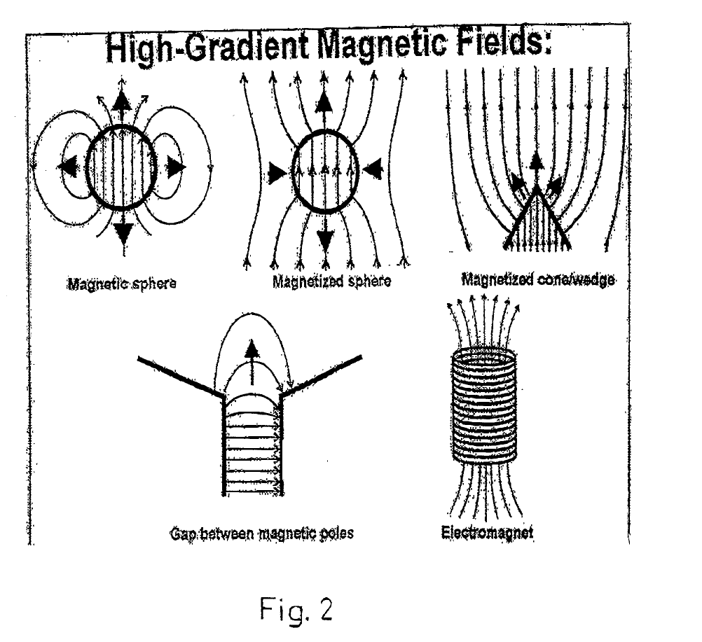

FIG. 3 is a diagram of mass transfer across skin in a gradient magnetic field;

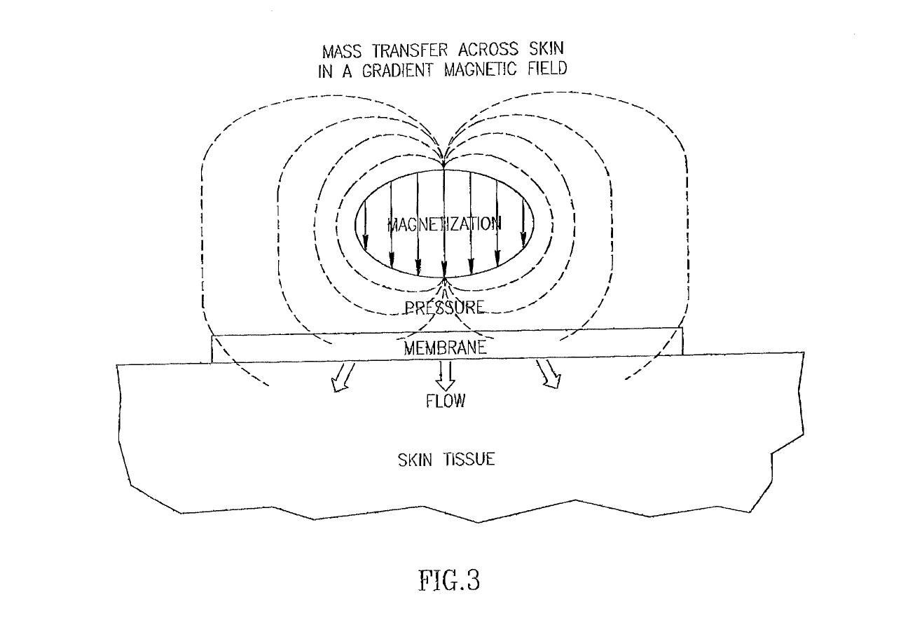

FIG. 4 shows a block diagram of the system of the present invention;

FIG. 5 shows a cross-section of an applicator pad of the present invention:

FIG. 6 shows various embodiments of the wedge of the present invention;

FIGS. 7a-b show a distribution pattern of the wedges of the present invention;

FIGS. 8a-b show waveforms for the high voltage generator;

FIG. 9 is a waveform for the electrical generator;

FIG. 10 is the response curve of the electromagnet; and

FIG. 11 is a block diagram of a test cell model.

### DETAILED DESCRIPTION OF THE PREFERRED EMBODIMENTS

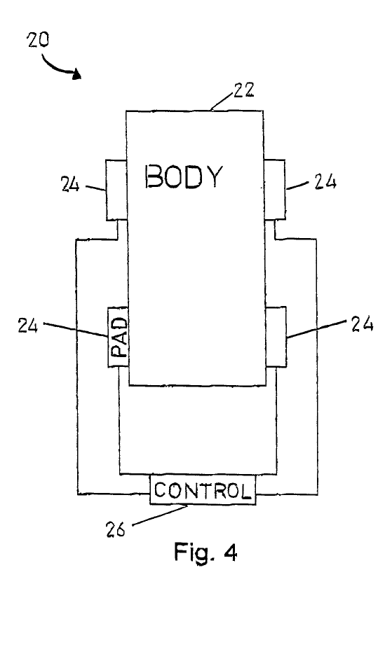

Referring now to FIG. 4, there is shown a block diagram of transdermal magneto drug delivery system 20 constructed and operated in accordance with the principles of the present invention. The transdermal magnetic drug delivery system of and method the present invention can deliver multiple drugs in a controlled manner using a variety of active transport methods including magnetophoresis, iontophoresis, sonophoresis, photophoresis and others. The combined use of these methods not only enhances transport but also allows for the optimum parameters for drug delivery to be realized. The drugs may be tagged with a magnetic carrier to increase their susceptibility to magnetophoresis and to provide a method for tracking their absorption.

In a preferred embodiment, system 20 is configured so that body part 22 has applied thereto four applicator pads 24, each of which is controlled by control block 26. Each of applicator pads 24 provides delivery of at least one type of substance to body part 22. Each pad 24 may provide delivery of a different substance and may use different methods of active transport delivery, including magnetophoresis, eletrophoresis, sonophoresis, light, or laser in singular or in combination. This approach enables multi-channel drug delivery providing integrated modes of delivery and therapeutic remimes.

In order to benefit from the magnetokinetic transport effect described in the Background, the present inventors have found that when the magnet is designed in a wedge configuration, the gradient and magnetic flux concentration is increased by a High Gradient Magnetic Field (HGMF). The general wedge design is based upon scientific literature (Kutznetsov O A et al. Planta 1996; 198(1):87-94, Multer-Rurhholtz W. et al U.S. Pat. No. DE 197 06 617 C1 (Feb. 2, 1997) for creating a “magnetic species” for drug delivery, the hypothesis being to develop optimum magnetokinetic forces by employing HGMFs for magnetophoresis with a ferromagnetic wedge. The magnetic design parameters call for the application of a “magnetoconductive wedge” consisting of soft steel, iron or other magneto-conductive substances to be used in conjunction with a strategic placement of high quality permanent Neobedium-Iron Boron magnet. The magnetic gradient can be increased by various techniques and methods such as the use of ferromagnetic rods and various types of wedge designs.

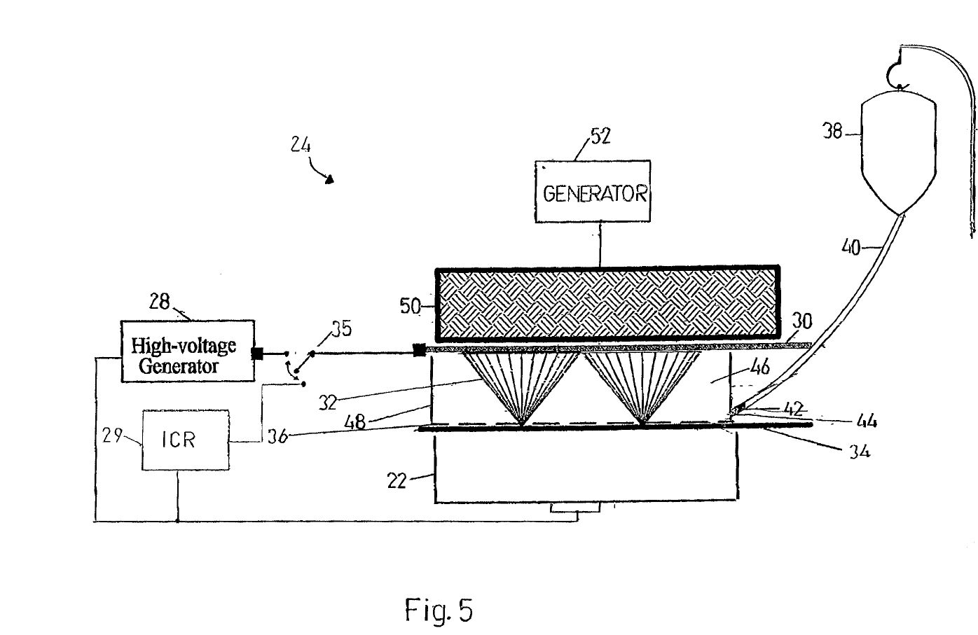

The construction of applicator pad 24 is shown in detail in FIG. 5. Wedge micro-electrodes 32, which serve as the anode electrode, are connected either to high voltage generator (HVG) 28, or to iontophoresis current reference (ICR) 29. HVG 28 provides electrical voltage to silver plate 30, which has a galvanic connection to wedge micro-electrodes 2. Wedge micro-electrodes 32 are in pin-point contact with skin 34 on body part 22, causing the skin to act as a cathode electrode. Iontophoresis current reference 29 provides an appropriate electric current between the anode and cathode electrodes. Switch 35 is provided to enable selection of HVG 28 or ICR 29, or a sequential combination of their outputs.

Alternatively, there may be an electroconductive membrane 36 between wedge micro-electrodes 32 and skin 34. The pulse sent by HVG 28 to wedges 32 causes controlled electroporation in skin 34, so as to allow for drug delivery across the skin surface. Drug solution is supplied from reservoir 38, via tubing 40 to valve 42, which when open allows solution to flow through Inlet 44 to the interior of cell 46.

In another alternative embodiment, valve 42 is integrated into the control function provided by control block 26, so that a titration feed system is achieved. A biosensor may be provided as disclosed in U.S. Pat. No. 5,983,134 to Ostrow, et al., a co-inventor of the present invention. The biosensor provides information to control block 26 on the rate of absorption and the need for further drug delivery.

Cell 46 is defined by walls 48 on the sides, which form a hermetic seal with skin 34 so as to prevent leakage of drug solution, and plate 30 on the top. Alternately, cell 46 may have a bottom surface provided by electroconductive or piezoelectric membrane 36.

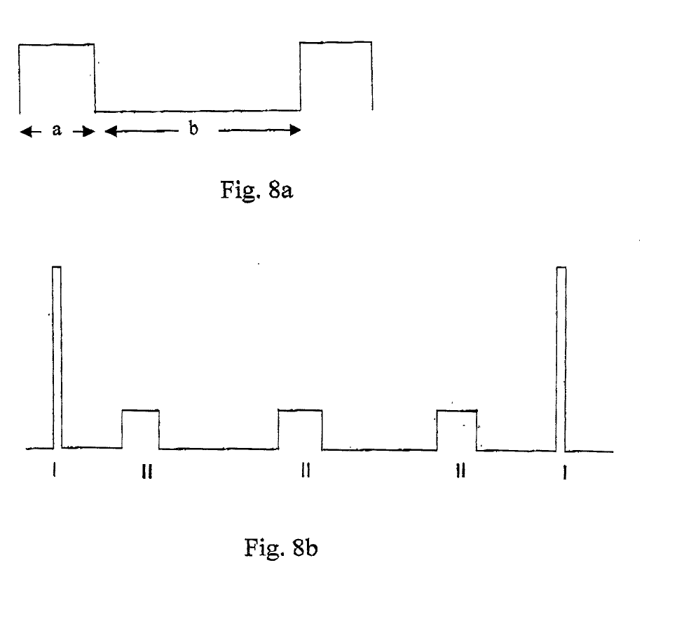

In accordance with the principles of the present invention, magnetophoresis occurs in combination with electroporation. The magnetic field is provided by electromagnet 50, which derives its electricity from electrical generator 52. This provides a solution to the prior art problem described in the Background of the Invention, in which the transient quality of the artificial pores is described. Since the electroporation is occurring in combination with the magnetophoresis and/or other driving forces, the pores are constantly being formed and the electroporation effect is not lost. This can be better appreciated with reference to the discussion of FIG. 8b, which shows a waveform for combined electroporation and electrophoresis,

The magnetic field may be provided by electromagnet 50 alone or in combination with a permanent magnet or may be provided by a permanent magnet by itself. The ability of electromagnet to adjust the variable output configuration is of primary importance, when adapted to the electrochemical and physical behavior of the moving particles as well as the diamagnetic properties of the solution. The engineering of the electromagnet and its generator creates an “IEMP drug delivery species” which is defined as modulation of the output of specific matching magnetic field frequencies that resonate at varying wavelengths to match the specific attenuation points relevant to the molecules of drugs dissolved in a solute according to their specific magnetic susceptibilities.

The following are possible parameters of electromagnet 50, by way of example:

a, Operating Voltage: 60 Volts-120V

b. Amperage: up to 8A

c. Magnetic field: up to 0.9 Tesla

d. Coil inductance 53 Henry

e. Coil resistance 17.5 Ohm

f. Wire diameter is 0.71 mm Generator 52 is provided with the following capabilities;

i. Very high current output.

ii. Continuous and interrupted mode.

iii. Alternate positive and negative signal.

iv. Adjustable ratio between ON time and OFF time.

The development of the electromagnet for transdermal drug delivery is based upon the theory that all drugs have attenuation points where they can be influenced by magnetic fields. Two basic mechanisms involving the effects of magnetic fields are involved in the mechanism influencing drug transport in a model system:

1. passive diffusion of molecules due to influence of the magnetic field induced, and

2. the mechanical stress at the border of materials with different magnetic susceptibilities.

Both mechanisms are responsible for the change in transportation of drug molecules under the Influence of a magnetic field. To accomplish the proper conditions for drug delivery employing magnetic fields, the design of a magnetic (MF) or electromagnetic (EMF) species must provide a magnetic field that can either influence or effect the velocity of molecules according to their attenuation points and magnetic susceptibilities. This is of paramount importance. For example, the rise time of the magnetic field generator has to match the molecular weight and attenuate to the magnetic susceptibility of the molecules of the drug used.

The average power of the electromagnet is

$$P = {\int }_{O}^{∞} V ( t ) I ( t ) ⅆ t \qquad ( 6 )$$

The above relates to the rise time of the pulses.

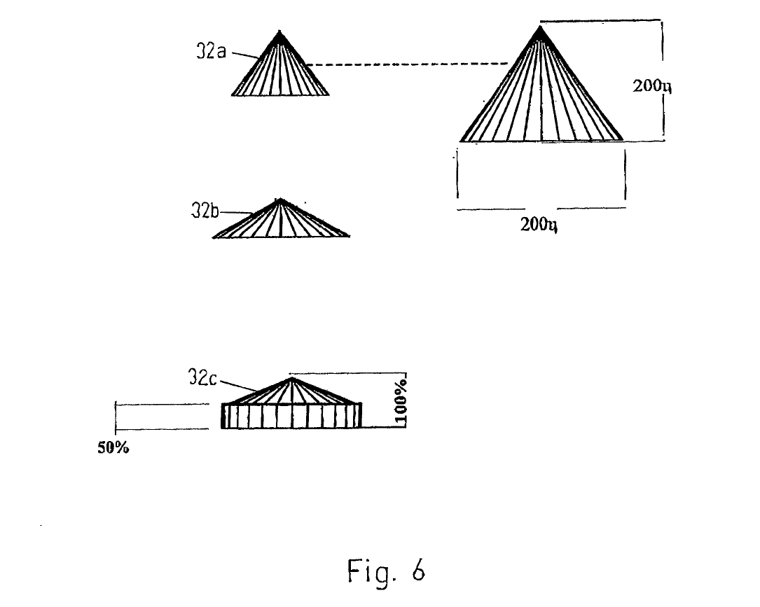

Wedge micro-electrodes 32 have a unique construction, as shown in FIG. 6. The wedge design includes a multitude of small substantially conical-shaped electrodes 32, arranged as a grid, which are attached to a conductive plate made, by way of example, from silver. The wedge micro-electrodes 32 are galvanically connected to plate 30. Wedges 32 are built with an apex angle of approximately 90 (32a)-120 (32b) degrees to create a focused gradient magnetic field where the HGMF beam is focused at the apex of the magnetic wedge. Wedge 32 is formed from soft Iron (Fe) with dimensions of approximately 200μ height and 200μ width. In a third embodiment the wedge may be of a combined type 32c in which the lower 50% of the height is at a 90 degree angle from the plate, while the upper 50% is comprised of a wedge formed at a 120 degree angle in a similar fashion to wedge 32b.

Alternatively, wedge micro-electrode 32 may be replaced by a nut-shaped wedge design, made from a circumferential hollowed steel-nut with four engraved grooved slots as a channel to allow flow of drugs through the wedge.

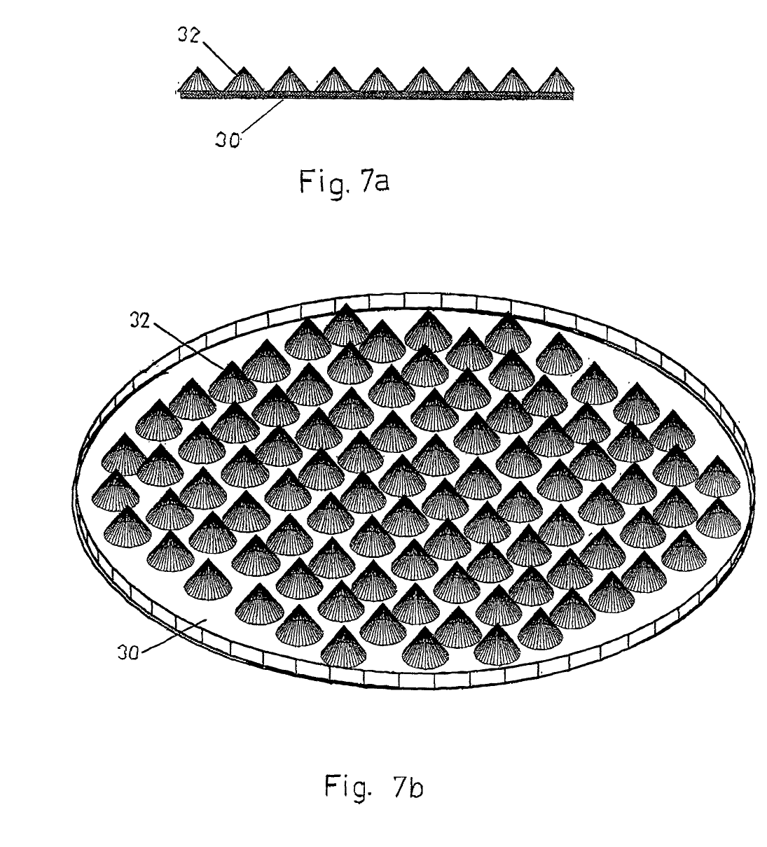

A preferred distribution pattern for wedges 32 is shown in FIGS. 7a-b. In FIG. 7a, there is shown a cross-section of the plate 30 with wedges 32 thereon. In FIG. 7b there is shown a top view of the plate 30 with wedges 32 thereon.

The wedge micro-electrode 32 of the present invention has been provided so as not only to focus HGMFs, but also as a source of controlled electroporation, in direct contrast to the flat electrodes previously discussed in the Background to the Invention. Unlike the flat electrodes used with prior art methods, the pin-point contact of the wedge micro-electrode 32 allows a higher current density to be achieved at the skin intefere at a lower voltage.

Referring now to FIG. 8a, there is shown a waveform for the pulses generated by high voltage generator 28 and delivered to wedges 32 for causing electroporation. The pulse durations “a” are approximately between 100μ sec to 1 msec, with pulse intervals “b” lasting approximately between 10 msec and up to 1 sec.

Wedge micro-electrodes 32 have a dual function, in providing both pulses for electroporation and for electrophoresis, when electrophoresis is one of the chosen drivers. FIG. 8b shows the composite waveform generated by superposition of the individual waveforms produced by HVG 28 and iontophoresis current reference 29. Pulses I are provided by HVG 28 as high voltage, short duration pulses which provide electroporation. Pulses II are provided by ionotophoresis current reference 29 as lower voltage, longer duration pulses which provide electrophoresis. The development of this waveform, in which Pulses II immediately follow Pulses I, enables drug delivery to take place effectively, because the electroporation effect has not been lost and the drug delivery takes place before the skin pores close again.

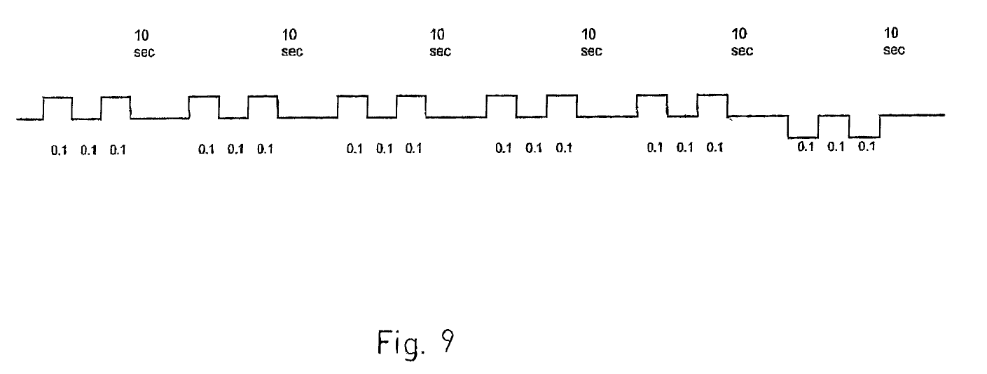

FIG. 9 shows a waveform for electromagnet 50 as generated by generator 52. The waveform consists of double pulse “c”, with each pulse being approximately 50 -100 msec long and being separated by 100 msec, and a pulse interval “d” of approximately 10 sec. Double pulse “c”, followed by pulse interval “d” are repeated for five sequences in the positive direction and one inversion sequence of a negative double pulse. Pulse Interval “d” must be at least 100 msec for changing polarity. The changing of the polarity mimics the effect of oscillation by preventing the skin from adapting to the magnetic field. The oscillation effect causes a pumping action to “push” the drug molecules through the membrane.

The double pulse magnetic field is employed to overcome impedance of the skin. The first pulse is designed for the initiation of the ion, whereas the second pulse accomplishes movement of the ion. The two signals, although independent, are seen as one wide homogenous signal that increases the effects of molecular transport in or through a solution, membrane or biological barrier (ie. Skin or cells). Additionally, the use of a double pulse in place of a single wide pulse saves energy.

In accordance with the principles of the present invention the waveforms of FIGS. 8 and 9 may be applied in a combined fashion so as to provide simultaneous electrophoresis, magnetophoresis and electroporation. It will be appreciated that this combination can be extended to include further modes of delivery, including photophoresis, sonophoresis and others, alone or in combination.

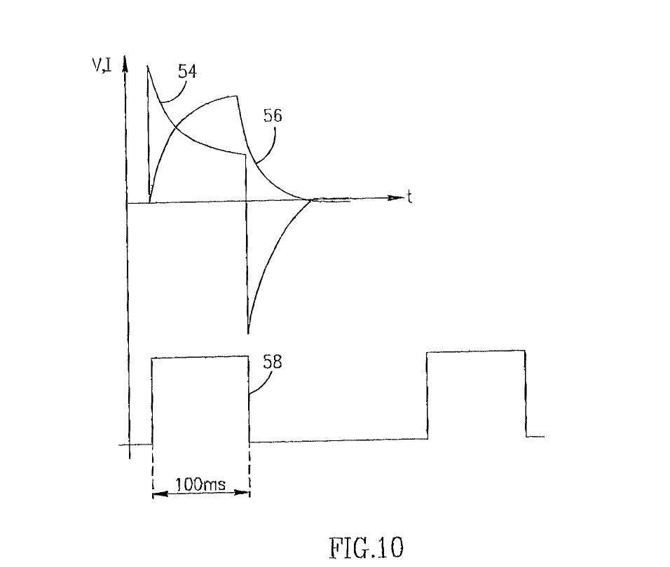

FIG. 10 shows the response curve of the electromagnet plotted as V (curve 54) and I (curve 56) versus time in response to the voltage pulses supplied by generator 52. The electromagnetic field established by the voltage pulses is repeatedly built and collapsed at a frequency known as the magnetic field frequency. It is necessary to tailor this frequency to the attenuation parameters of varying molecules comprising the drug substance so as to achieve the magnetokinetic effect.

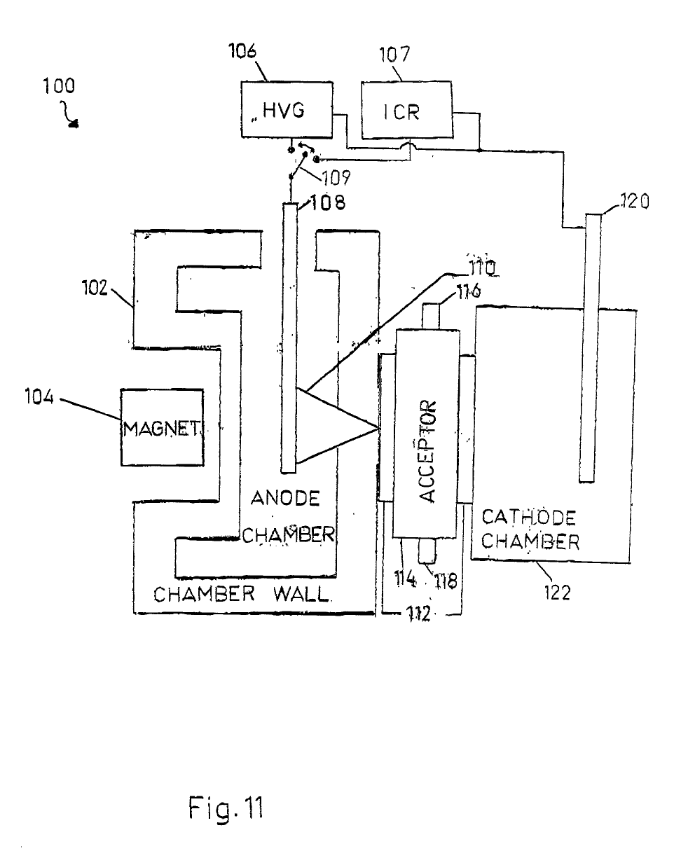

FIG. 11 is a block diagram of test cell model 100 for measuring transport efficiency of different drugs under various delivery modes in a laboratory setting. Test cell model 100 provides multiple treatment and drug delivery protocols for controlled targeted closed drug loop systems employing the magnetic susceptibilities if pharmaceutical compounds, substances and metals for drug delivery modulation with an intelligent feedback system.

Test cell model 100 is provided with an anode chamber housing 102, typically formed of plexiglass. Magnet 104, which may be an electromagnet, a permanent magnet or a combination thereof, is placed outside housing 102 and generates a magnetic field within housing 102. High voltage generator 106 provides a predetermined waveform to anode electrode 108, which has a galvanic connection to wedge micro-electrode 110. Alternatively, iontophoresis current reference 107 may provide an alternate waveform for electrophoresis. Switch 109 allows selection of the waveform output source for input to electrode 108.

Wedge micro-electrode 110 extends through the plexiglass of housing 102 to come in contact with a membrane 112 which is either skin or an artificial biological membrane.

In operation, drug solution from within housing 102 is driven across membrane 112 into acceptor 114. Acceptor 114 has inlet 116 and outlet 118 for providing a buffer solution, such as PBS (phosphate buffer solution) to flush out acceptor 114. PBS is collected from outlet 118 and drug concentration can be monitored to determine the efficiency of transport. Cathode electrode 120 is provided in cathode chamber 122 to complete the loop.

The transdermal magnetic drug delivery system of the present invention may be used for the exploitation of magnetic susceptibilities of substances for controlled, targeted drug delivery transdermally or internally in the body. This may be done by tagging, manipuiating or doping substances with a magnetic carrier having nutritional properties and little or no side effects. The magnetic carrier should be paramagnetic to enhance the substance's susceptibility to magnetophoresis and/or electromagnetophoresis, and to increase control and targeting abilities. This process may be called pharmaco-nutrification, and may be used to design drugs, without changing their compound formulation to be targeted to any specific anatomical area of the mammalian body. For example, drugs such as insulin may be tagged with vanadium, chromium or other mimetic substances that have both insulin-like qualities and are magnetically susceptible, for magneto transport transdermally or within the mammalian body. The tag may be used to direct the substance within the body to a specific organ or target area. Pharmaco-nutrification maybe accomplished using micronutrients that are magnetically susceptible such as iron, calcium, sodium, copper, nickel, zinc, boron, molybdenum and others for bonding to the substance.

Once the substance is tagged with the magnetic carrier, the substance becomes marked, and therefore can be used for non-invasive, intelligent bio-sensing and for tracking drug concentration in the skin and blood circulation with the use of magnetic fields to pick up the specific signal of the carrier. A biosensor may be used and built as disclosed in U.S. Pat. No. 5,983,134 to Ostrow et. al., a co-inventor of the present invention,

Compounds like DMSO and other solvents can be added to increase permeation and solubility of metals and drugs that have problems with solubility.

Therefore, the system of the present invention overcomes the deficiencies of prior art drug delivery systems, both those that do and those that do not employ magneto-transport.

Having described the invention with regard to certain specific embodiments thereof, it is to be understood that the description is not meant as a limitation, since further modifications may now suggest themselves to those skilled in the art, and it is intended to cover such modifications as fall within the scope of the appended claims.

We claim:

1. A system for transdermal magnetic delivery of a substance in solution to an acceptor, said system comprising:
at least one substance delivery means employing electroporation of the acceptor in the presence of a magnetic field, and also employing, in sequential fashion, a mode for active transport; and
means for controlling said at least one substance delivery means;
said system being operable to provide combined electroporation of the acceptor and active transport of the substance in solution, in a controlled fashion, thereby delivering the substance to the acceptor.

2. The system of claim 1 wherein said substance delivery means comprises;
means for providing electroporation of a membrane in contact with the acceptor;
means for providing active transport through said membrane;
means for developing a magnetic field, and
a holding device for holding the substance in contact with said membrane.

3. The system of claim 2 wherein said substance delivery means is provided as at least one applicator pad.

4. The system of claim 1 wherein said active transport mode comprises at least one of iontophoresis, electromagnetophoresis, sonophoresis, and photophoresis.

5. The system of claim 2 further comprising a reservoir from which the substance is provided to said holding device.

6. The system of claim 2 wherein said means for developing said magnetic field comprises at least one of a permanent magnet and an electromagnet.

7. The system of claim 6 wherein said electromagnet is provided with an electric field generator producing a first voltage waveform to said electromagnet.

8. The system of claim 7 wherein said first voltage waveform includes double pulses each of a duration between approximately 50 msec to approximately 100 msec, each pulse of said double pulses being separated from the other pulse by 100 msec and said double pulses being separated by intervals of between approximately 10 msec to approximately 10 sec.

9. The system of claim 8 wherein said double pulses are repeated 5 times in sequence having a positive polarity followed by one inverted double pulse having a negative polarity.

10. The system of claim 2 wherein said means for providing electroporation comprises:
a plurality of substantially conically-shaped micro-electrodes distributed across an electrically conductive plate, said plate having a galvanic connection with said electrodes, said electrodes having a galvanic connection with said membrane,
a voltage generator connected to said conductive plate for providing a second voltage waveform to said electrodes through said plate.

11. The system of claim 10 wherein said plurality of substantially conically-shaped micro-electrodes are arranged in a grid across said electrically conductive plate.

12. The system of claim 10 wherein said conductive plate is silver.

13. The system of claim 10 wherein said conductive plate is approximately 25 mm in diameter.

14. The system of claim 10 wherein said micro-electrodes are formed from iron.

15. The system of claim 10 wherein said micro-electrodes have dimensions of approximately 200 microns in height, and approximately 200 microns width.

16. The system of claim 10 wherein an angle is defined at the apex of said conically-shaped micro-electrodes, said angle being approximately within the range of 90-120 degrees.

17. The system of claim 10 wherein an angle is defined at the apex of said conically-shaped micro-electrodes, said angle being approximately 90 degrees.

18. The system of claim 10 wherein an angle is defined at the apex of said conically-shaped micro-electrodes, said angle being approximately 120 degrees.

19. The system of claim 10 wherein said conically-shaped micro-electrode is comprised of at least two sections, the lower section being at a right angle to said plate and the upper section being conically-shaped and having an angle defined at its apex, said angle being approximately within the range of 90-120 degrees.

20. The system of claim 10 further comprising a ferromagnetic element in contact with said substance delivery means for increasing the magnetic gradient.

21. The system of claim 20 wherein said element is provided as at least one of rods and wedges.

22. The system of claim 10 wherein said second voltage waveform comprises pulses of between approximately 100 μsec to approximately 1 msec duration and intervals of between approximately 10 msec to approximately 1 sec.

23. The system of claim 10 further comprising an electroconductive membrane providing galvanic contact between said plurality of electrodes and said membrane.

24. The system of claim 1 wherein said means for providing electroporation comprise a circumferential hollow steel-nut with multiple engraved grooved slots forming channels enabling flow of the substance through said steel-nut.

25. The system of claim 2 further comprising a biosensor.

26. The system of claim 1 further comprising at least one of solvents and lipolytic carriers in the substance to increase permeability and solubility.

27. A method for transdermal magnetic delivery of a substance in solution to an acceptor, said method comprising the steps of;
providing at least one substance delivery means employing electroporation of the acceptor in the presence of a magnetic field, and also employing, in sequential fashion, a mode for active transport; and
controlling said at least one substance delivery means,
said method providing combined electroporation of the acceptor and active transport of said substance in solution, in a controlled fashion, thereby delivering the substance to the acceptor.

28. The method of claim 27 further comprising, prior to the step of providing electroporation, the step of modifying the substance to incorporate a carrier component having a magnetic susceptibility.

29. The method of claim 28 wherein said step of modifying the substance comprises pharmaco-nutrification.

30. The method of claim 28 wherein said carrier component is a micronutrient.

31. The method of claim 30 wherein said micronutrient is from the group of iron, calcium, sodium, copper, nickel, zinc, boron and molybdenum.

32. The method of claim 28 wherein said carrier component is a mimetic substance.

33. The method of claim 32 wherein said mimetic substance is from the group of vanadium and chromium.

34. The method of claim 28 further comprising the step of biosensing substance concentration by detecting said carrier component as a marker.

35. The method of claim 28 wherein said stop of modifying further includes the step of tracking substance concentration in at least one of the skin and the blood circulation.

36. The method of claim 28 wherein said step of modifying further includes the step of targeting the substance to a specific target area.

37. A test cell system for determining optimum parameters for achieving transdermal magnetic delivery of a substance in solution, said system comprising:
an acceptor having a solution therein,
a membrane in contact with said acceptor cell;
a donor cell for holding the substance in contact with said membrane;
means for providing electroporation of said membrane;
means for providing a magnetic field in proximity to said membrane;
means for providing a mode of active transport;
means for controlling said electroporation means, said magnetic field means, and said active transport mode;
said system providing combined electroporation of said membrane and magnetophoresis of said substance in solution, and active transport, in a controlled fashion, thereby delivering the substance to the solution in said acceptor cell.

38. A multi-channel system for transdermnal magnetic delivery of at least one substance in solution to an acceptor, said system comprising;
a multiple of substance delivery means each employing electroporation of the acceptor in the presence of a magnetic field, and also employing, in sequential fashion, at least one mode for active transport, and
means for controlling said multiple substance delivery means,
said system being operable to provide combined electroporation of the acceptor and active transport of the at least one substance in solution, in a controlled fashion, thereby delivering the substance to the acceptor.

39. The system of claim 38 wherein the substance is modified to incorporate a carrier component having a magnetic susceptibility.

40. The method of claim 39 further comprising a biosensor for biosensing substance concentration by detecting said carrier component as a marker.
